(19) United States
(12) Patent Application Publication
(10) Pub. No.: US 2003/0060824 A1
(43) Pub. Date: Mar. 27, 2003

(54) Linking rod for spinal instrumentation

(76) Inventors: Guy Viart, Saint Leger (FR); Jean-Paul Steib, Strasbourg (FR); Emeric Gallard, Paris (FR)

Correspondence Address: GREENBLUM & BERNSTEIN, P.L.C., 1950 ROLAND CLARKE PLACE, RESTON, VA, 20191, US

(21) Appl. No.: 10/169,745

(22) PCT Filed: Jan. 12, 2001
(86) PCT No.: PCT/FR01/00097

(30) Foreign Application Priority Data
Jan. 18, 2000 (FR) 00/00563

(51) Int. Cl. A61B 17/56

(52) U.S. Cl. 606/61

(57) ABSTRACT

The invention concerns a longitudinal linking rod for spinal instrumentation, produced in a malleable alloy whereof the ultimate elongation A %, in tensile test, is higher than 20%.

The present invention relates to a linking rod for spinal instrumentation, consisting mainly of anchoring screws, fixed in each vertebra, of hooks or of clips, or of similar elements mounted on the anchoring screws.

Various types of spinal instrumentation or devices are already known, such as those by the name ISOLA, these allowing the spine or spinal column of a patient suffering, for example, from a scoliosis to be straightened and supported.

The vertebral anchoring elements, consisting for example of screws and hooks, are fixed to the pedicles or the plates of each vertebra of the deformed spinal segment, before receiving a linking rod.

Each spinal instrumentation linking rod is bent by the surgeon, away from the operating area, according to the desired vertebral profile, in order to correct the deformed spinal segment.

The first and second bent rods are placed respectively on each vertebral anchoring element so as to join them together.

It has been found that placing a linking rod is difficult because of its rigid material, which does not allow it to easily follow the curvatures of the deformed spinal segment.

Thus, the rigid material of the linking rod requires the surgeon to use various instruments for bringing the vertebral anchoring elements of the linking rod closer together in order to insert it into each anchoring element.

It should be noted that the linkage obtained between the rods and the anchoring elements is the seat of parasitic stresses which will subsequently entail a substantial risk of the spinal device or instrumentation failing.

Furthermore, bringing the vertebral anchoring elements closer toward each of the linking rods may again entail in situ bending of each rod in order to perfect the correction. This in situ bending, that is to say bending in the operating area or on the patient, is made very difficult because of the mechanical properties (high rigidity) of the rod.

In certain cases of substantial spinal deformations of the scoliosis type, the surgeon must make additional corrections which consist in making one of the linking rods rotate about its longitudinal axis.

This technique, called “CD technique”, may be defective in a number of cases, namely:

double thoracic scolioses;

thoraco-lumbar scolioses;

deviations extending to the sacrum.

This CD technique is also ineffective in thoracic scolioses with a slight frontal deformation. This is because, without frontal deformation the straight linking rod rotated about its axis causes no modification of the axial plane.

Finally, this CD technique is no longer applicable in cases of serious scolioses without the use of complementary rods which are complicated to install.

It will be noted that rotation of the linking rod about its longitudinal axis takes en bloc the entire spinal segment to be corrected, which may entail frontal equilibrium problems difficult to solve.

The object of the present invention is to provide a linking rod for spinal instrumentation, having mechanical properties allowing it to deform in situ, that is to say in the operating room, without causing the drawbacks of the prior art described above.

The description which follows, in regard to the appended drawings, given by way of nonlimiting examples, will allow the invention, the features that it has and the advantages that it can provide to be better understood.

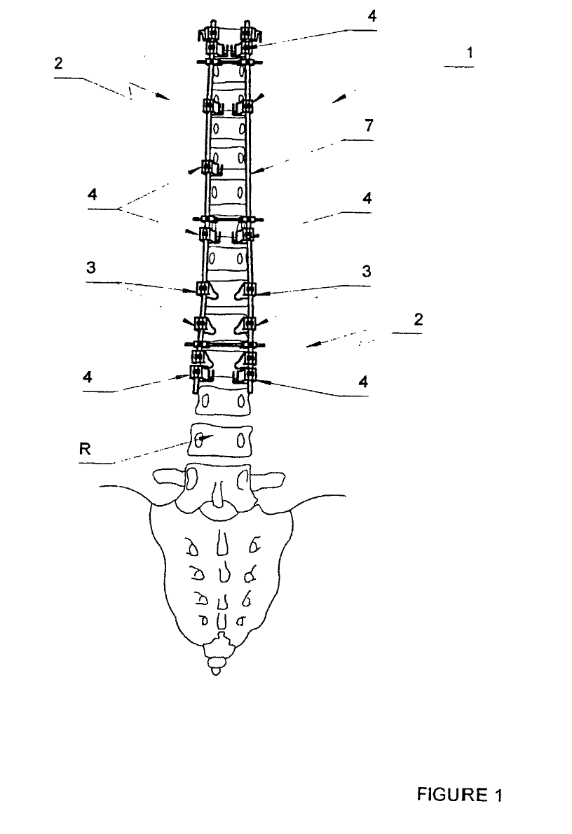

FIG. 1 is a view in the frontal plane illustrating the rear face of a spinal segment to which a spinal instrumentation comprising in situ deformable linking rods according to the present invention is fixed.

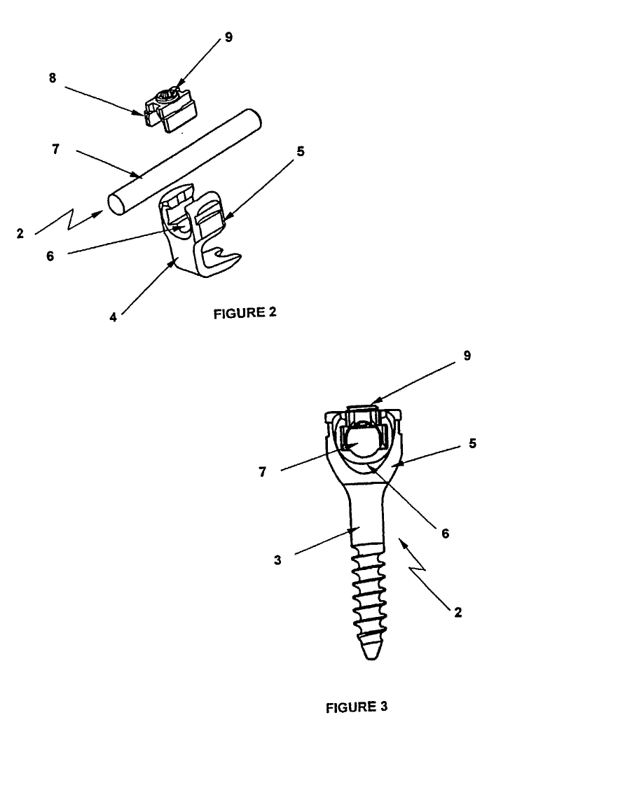

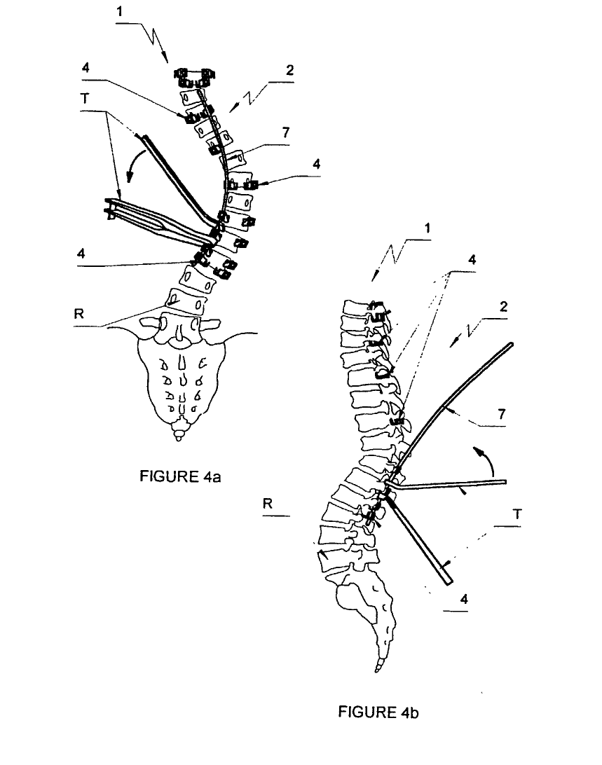

FIGS. 2 and 3 are exploded perspective views showing examples of spinal instrumentations capable of receiving the linking rod according to the present invention.

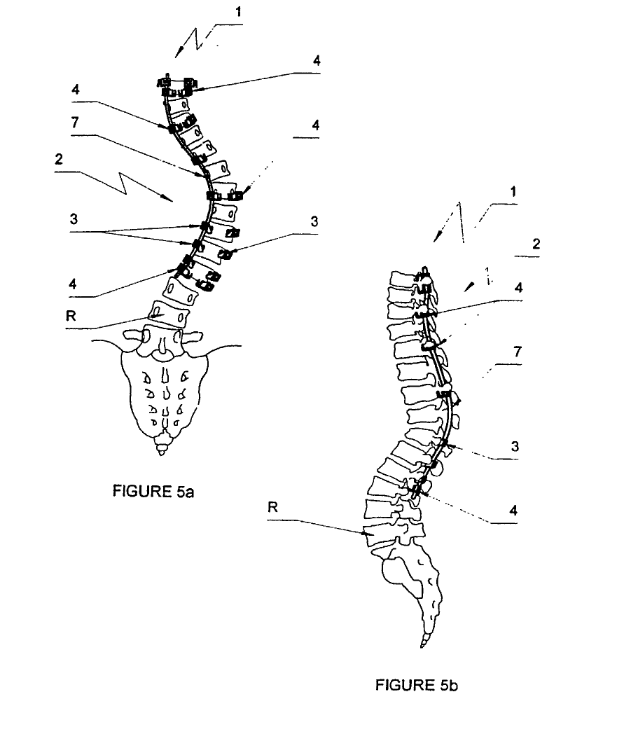

FIGS. 4a and 4b are views representing, in the frontal plane and in the sagittal plane, the in situ installation of a linking rod on the vertebral anchoring elements of the spinal instrumentation.

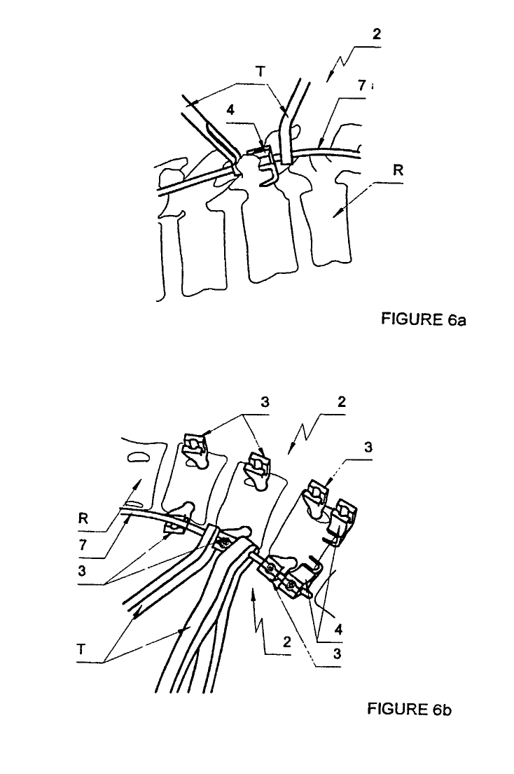

FIGS. 5a and 5b are views illustrating, in the frontal plane and in the sagittal plane, a linking rod which matches the deformed profile of the spinal segment to be corrected.

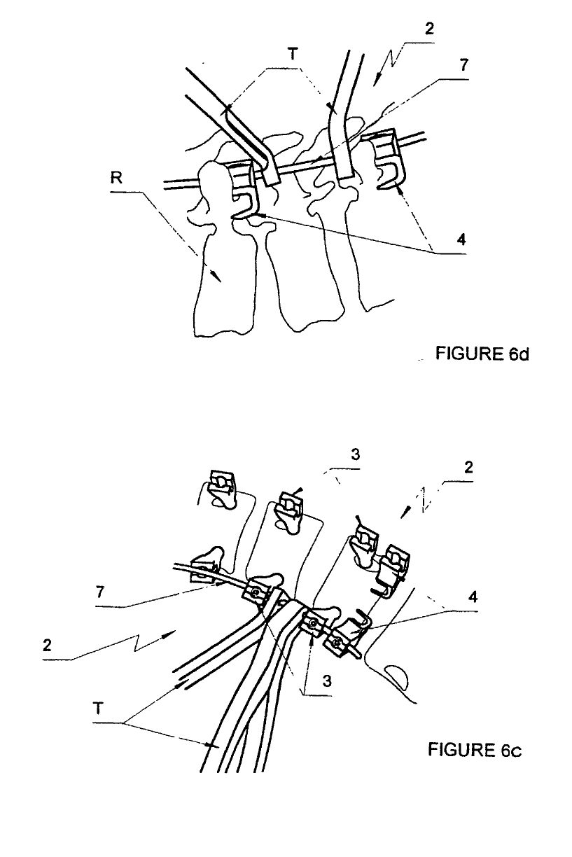

FIGS. 6a to 6d are views showing the in situ modeling of a linking rod in order to correct the deformation of the spinal segment.

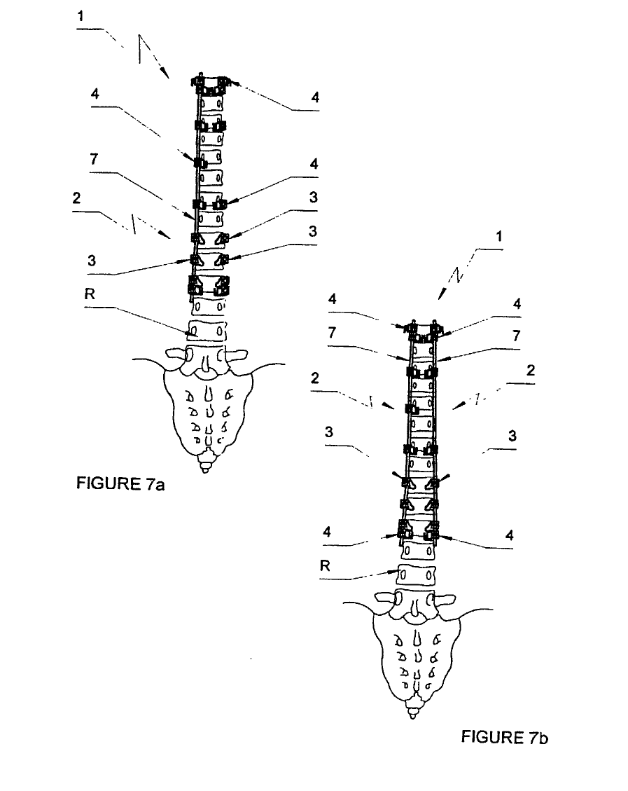

FIGS. 7a and 7b are views showing the corrected spinal segment after deformation of the linking rod.

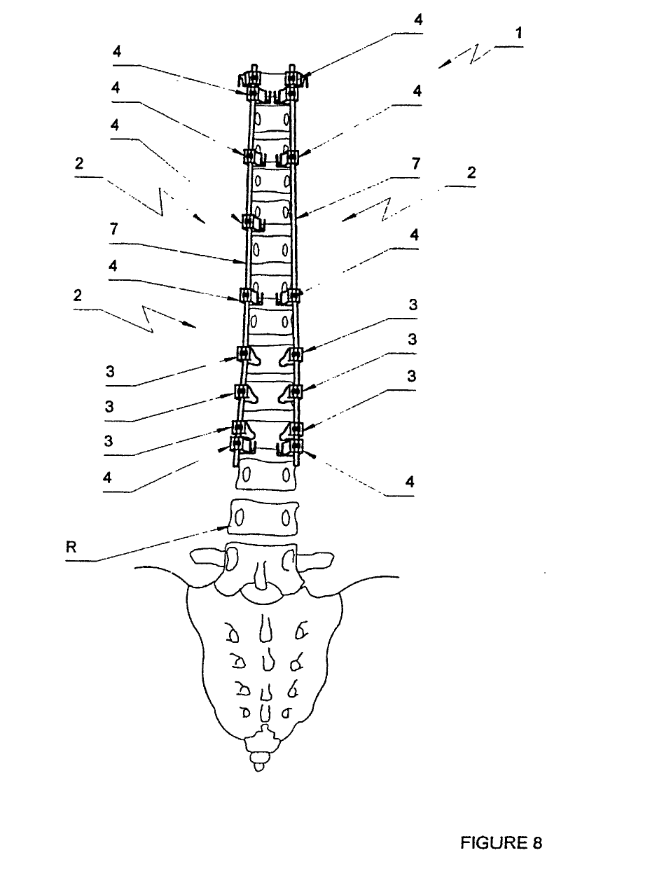

FIG. 8 is a view illustrating the spinal segment corrected by means of two linking rods according to the invention.

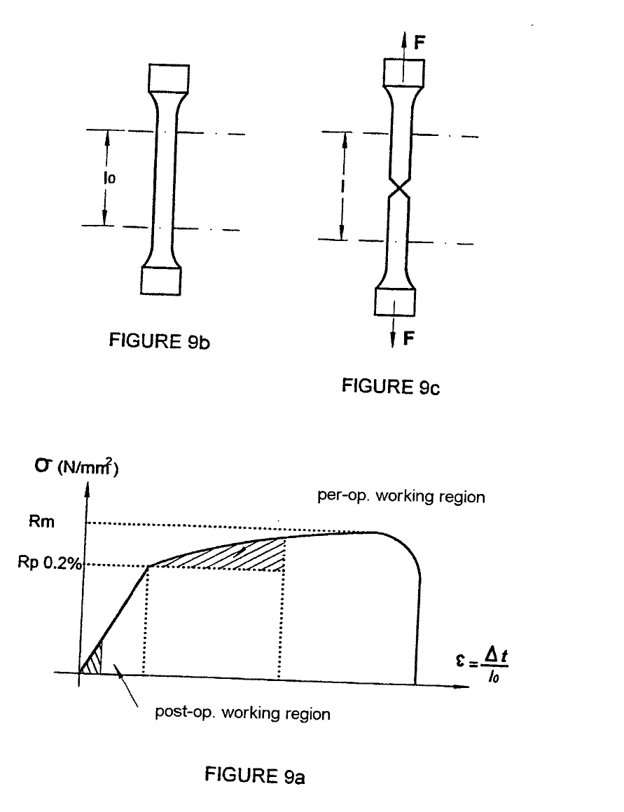

FIGS. 9a to 9c are diagrams showing the elongation to break characteristics in a tensile test on the linking rod according to the invention.

FIGS. 1 and 3 show a spinal instrumentation 1 repeating all the features of those described in patent EP 0 773 746 belonging to the applicant.

This spinal instrumentation 1 consists of vertebral anchoring elements 2 which are fixed to the vertebrae of the spinal segment R to be corrected, either via screws 3 or by means of hooks 4.

Each anchoring element 2 comprises an open body 5 having a U-shaped profile intended to receive, in its circularly arcuate bottom 6, a linking rod 7.

The open body 5 receives, by sliding against the bottom 6 of the U, a clip 8 having clamping means 8 which apply radial clamping pressure to the linking rod 7.

When the anchoring elements 2 are fixed to the vertebral bodies of the spinal segment R, the surgeon then carries out the in situ installation and modeling of the linking rod 7.

Thus, the linking rod 7 is curved or modeled so as to be able to be inserted into the open bodies 5 of each anchoring element 2, in order to bear against the bottom 6 of the latter.

Flexural deformation of the linking rod 7 is performed using instruments T, allowing said rod to be curved or modeled in the frontal plane and then in the sagittal plane of the deformed spinal segment R, so as to insert said rod into the open body 5 of the adjacent anchoring element 2 (FIGS. 4a and 4b).

Insertion of the linking rod 7 is found to be completed when the latter matches, after the first progressive modeling, the curvatures of the deformed spinal segment R. This plastic deformation of the linking rod 7 according to the profile of the deformed spinal segment R makes it possible to avoid any mechanical stress between said rod and the anchoring elements 2 which have been fixed beforehand in the vertebral bodies (FIGS. 5a and 5b).

The linking rod 7 is linked to each anchoring element 2 via clips 8 which are inserted into each open body 5. Fitting the clips 8 is designed to allow the linking rod 7 to have freedom of movement in terms of translation and rotation inside each anchoring element 2.

The spinal segment R is corrected by various progressive modeling actions on the linking rod 7 by means of the instruments T. These in situ modeling actions for correcting the spinal segment R are performed by the surgeon who applies, by means of the instruments T, forces which plastically deform the linking rod 7.

The plastic deformations of the linking rod 7 are produced, according to the correction to be applied to the spinal segment R, on each side of the same anchoring element 2 (FIGS. 6a and 6b).

The plastic deformations of the linking rod 7 may also be produced between two adjacent anchoring elements 2 (FIGS. 6c and 6d), in order to be able to provide the necessary correction to the spinal segment R.

Thus, the second progressive modeling of the linking rod 7 results in the spinal segment R undergoing a correction which is balanced in the frontal plane and sagittal plane (FIGS. 7a and 7b).

The surgeon will then proceed in the same way in order to install a second linking rod 7 which will supplement the modeling actions performed on the first linking rod in order to perfect the correction of the spinal segment R (FIG. 8).

The linking rod 7 is modeled by plastic deformation thanks to the chemical composition of the alloy and to its various metallurgical treatments which make it possible to obtain particularly advantageous mechanical properties.

Thus, the linking rod 7 is made of a rapidly quenched austenitic stainless steel which is very malleable in order to allow the first and second in situ progressive modeling operations.

This is because the linking rod 7 is obtained from an alloy which consists, in combination, of the following elements:

carbon (C);

silicon (Si);

manganese (Mg);

sulfur (S);

phosphorus (P);

nickel (Ni);

chromium (Cr);

molybdenum (Mo);

copper (Cu);

iron (Fe);

nitrogen (N).

The content as a percentage of each element for forming the alloy is:

<0.03 of carbon (C);

<0.75 of silicon (Si);

<2 of manganese (Mg);

<0.01 of sulfur (S);

<0.025 of phosphorus (P);

13<<15 of nickel (Ni);

17<<19 of chromium (Cr);

2.25<<3 of molybdenum (Mo);

<0.5 of copper (Cu);

BALANCE of iron (Fe);

<0.1 nitrogen (N).

The linking rod 7 may also be produced in a grade-2 titanium alloy, which allows the first and second in situ modeling operations on said rod in order to correct the spinal segment R.

The linking rod 7 obtained from a titanium alloy consists, in combination, of the following elements:

carbon (C);

iron (Fe);

hydrogen (H);

nitrogen (N);

oxygen (O);

titanium (Ti)

The content as a percentage of each element for forming the alloy is:

<0.1 of carbon (C);

<0.3 of iron (Fe);

<0.0125 of hydrogen (H);

<0.03 of nitrogen (N);

<0.25 of oxygen (O);

BALANCE of titanium (Ti).

It should be noted that the composition of the alloys based on stainless steel or on titanium complies, on the one hand, with the desired mechanical properties and, on the other hand, with the standards for the use of stainless steel or titanium alloys in surgical implants.

Furthermore, it is also necessary that the linking rod 7 made in an alloy based on stainless steel, have an elongation at break A % in the tensile test which has to be greater than 40% (FIGS. 9a to 9c).

However, the linking rod 7 made in a titanium-based alloy has an elongation at break A % in the tensile test which has to be greater than 20% (FIGS. 9a to 9c).

It should moreover be understood that the above description has been given merely as an example and that it in no way limits the scope of the invention, it not being outside the scope thereof to replace the embodiment details described by any other equivalent.

1. A spinal instrumentation comprising vertebral anchoring elements (2) consisting either of screws (3) or of hooks (4), clips (8) provided with clamping means (9) which cooperate with the anchoring elements (2) in order to prevent translational and rotational movement of a longitudinal linking rod (7), characterized in that the longitudinal linking rod (7) is a malleable rod which is inserted by progressive modeling into each anchoring element (2) in order, on the one hand, to match, after a first modeling operation, the profile of the deformed spinal segment (R) and to be linked by clips (8) to each anchoring element (2) in order to remain free to undergo translational and rotational movement, and, on the other hand, to correct the spinal segment (R) after a second modeling operation and to be immobilized inside said anchoring elements by the clips (8) provided with the clamping means (9).

2. The spinal instrumentation as claimed in claim 1, characterized in that the longitudinal linking rod (7) is a malleable rod which is modeled by means of bending instruments (T).

3. The spinal instrumentation as claimed in claim 1, characterized in that the longitudinal linking rod (7) is made of stainless steel, of which the elongation to break characteristic, A %, in the tensile test is greater than 40%.

4. The spinal instrumentation as claimed in claim 1, characterized in that the longitudinal linking rod (7) is made of titanium, of which the elongation at break characteristic, A %, in the tensile test is greater than 20%.

5. The spinal instrumentation as claimed in claim 1, characterized in that it comprises two malleable longitudinal linking rods (7).
